(19) United States
(12) Patent Application Publication
(10) Pub. No.: US 2011/0237971 A1
(43) Pub. Date: Sep. 29, 2011

(54) DISCRETE CHOICE MODELING USING NEURO-RESPONSE DATA

(75) Inventors: Anantha Pradeep, Berkeley, CA (US); Robert T. Knight, Berkeley, CA (US); Ramachandran Gurumoorthy, Berkeley, CA (US)

(73) Assignee: NeuroFocus, Inc., Berkeley, CA (US)

(21) Appl. No.: 12/731,868

(22) Filed: Mar. 25, 2010

(51) Int. Cl. A61B 5/04; A61B 5/00

(52) U.S. Cl. 600/544; 600/300

(57) ABSTRACT

A system obtains neuro-response data as well as survey based data during discrete choice modeling to evaluate subject decision making processes. A discrete choice model evaluates a decision made by a subject as a function of multiple variables. Neuro-response data vectors and orthogonal survey based data vectors are weighted and combined to generate multi-dimensional vectors. The multi-dimensional vectors are used to estimate the effectiveness of changing particular variables in modifying subject behavior.

### TECHNICAL FIELD

The present disclosure relates to using neuro-response data to perform discrete choice modeling.

### DESCRIPTION OF RELATED ART

Conventional systems for performing discrete choice modeling are limited. Some conventional systems provide subjects with sets of choices to evaluate the contribution of multiple variables in subject decision making processes. Results from post-articulation analyzers, manual language selection instruments, and/or survey-based language analysis are evaluated to determine the contribution of particular variables. However, conventional systems are subject to brain pattern, semantic, syntactic, metaphorical, cultural, and interpretive errors that prevent accurate and repeatable analyses.

Consequently, it is desirable to provide improved methods and apparatus for performing discrete choice modeling that uses neuro-response data such as central nervous system, autonomic nervous system, and effector system measurements along with survey based data.

### BRIEF DESCRIPTION OF THE DRAWINGS

The disclosure may best be understood by reference to the following description taken in conjunction with the accompanying drawings, which illustrate particular example embodiments.

FIG. 1 illustrates one example of a system for performing discrete choice modeling analysis using neuro-response data.

FIG. 2 illustrates survey based scores for discrete choice modeling.

FIG. 3 illustrates neuro-response based scores for discrete choice modeling.

FIG. 4 illustrates combination or survey base scores and neuro-response based scores.

FIG. 5 illustrates examples of reports that can be generated.

FIG. 6 illustrates one example of technique for performing discrete choice modeling.

FIG. 7 provides one example of a system that can be used to implement one or more mechanisms.

### DESCRIPTION OF PARTICULAR EMBODIMENTS

Reference will now be made in detail to some specific examples of the invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

For example, the techniques and mechanisms of the present invention will be described in the context of particular types of data such as central nervous system, autonomic nervous system, and effector data. However, it should be noted that the techniques and mechanisms of the present invention apply to a variety of different types of data. It should be noted that various mechanisms and techniques can be applied to any type of stimuli. In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present invention unless otherwise noted. Furthermore, the techniques and mechanisms of the present invention will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

Overview

A system obtains neuro-response data as well as survey based data during discrete choice modeling to evaluate subject decision making processes. A discrete choice model evaluates a decision made by a subject as a function of multiple variables. Neuro-response data vectors and orthogonal survey based data vectors are weighted and combined to generate multi-dimensional vectors. The multi-dimensional vectors are used to estimate the effectiveness of changing particular variables in modifying subject behavior.

### EXAMPLE EMBODIMENTS

Discrete choice modeling (DCM) is a mechanism for evaluating decision making processes. Subjects are provided with a finite set of exhaustive and mutually exclusive choices. Survey based responses are used to evaluate decisions and responses are correlated with attributes of subjects making the decisions. For example, the choice of what beverage a person buys may be statistically related to socioeconomic and demographic factors. The decision to market or improve a particular feature on an appliance, e.g. the energy savings, the quality, or the large capacity, can be made based on the impact of various features on subject decision making processes. These decision making processes are often evaluated using survey based discrete choice models. In some instances, the models estimate the probability that subjects having particular characteristics will choose a particular alternative. The models can also be used to forecast how subject behavior will be affected when attributes of the alternatives change.

Discrete choice modeling has conventionally been performed using mechanisms such as survey based responses and statistical data. Results from post-articulation analyzers, manual language selection instruments, and/or survey-based language analysis are evaluated to determine the contribution of particular variables. However, conventional systems are subject to brain pattern, semantic, syntactic, metaphorical, cultural, and interpretive errors that prevent accurate and repeatable analyses.

Some efforts have been made to use neuro-response data to perform discrete choice modeling (DCM). Neuro-response measurements such as central nervous system, autonomic nervous system, and effector measurements can be used to evaluate subjects during discrete choice modeling. Some examples of central nervous system measurement mechanisms include Functional Magnetic Resonance Imaging (fMRI), Electroencephalography (EEG), Magnetoencephlography (MEG), and Optical Imaging. Optical imaging can be used to measure the absorption or scattering of light related to concentration of chemicals in the brain or neurons associated with neuronal firing. MEG measures magnetic fields produced by electrical activity in the brain. fMRI measures blood oxygenation in the brain that correlates with increased neural activity. However, current implementations of fMRI have poor temporal resolution of few seconds. EEG measures electrical activity associated with post synaptic currents occurring in the milliseconds range. Subcranial EEG can measure electrical activity with the most accuracy, as the bone and dermal layers weaken transmission of a wide range of frequencies. Nonetheless, surface EEG provides a wealth of electrophysiological information if analyzed properly. Even portable EEG with dry electrodes provides a large amount of neuro-response information.

Autonomic nervous system measurement mechanisms include Electrocardiograms (EKG) and pupillary dilation, etc. Effector measurement mechanisms include Electrooculography (EOG), eye tracking, facial emotion encoding, reaction time etc.

Multiple modes and manifestations of precognitive neural signatures are blended with cognitive neural signatures and post cognitive neurophysiological manifestations to more accurately perform DCM. In some examples, autonomic nervous system measures are themselves used to validate central nervous system measures. Effector and behavior responses are blended and combined with other measures. According to various embodiments, central nervous system, autonomic nervous system, and effector system measurements are aggregated into a measurement that allows evaluation of subject decision making processes.

In some instances, users may collect both survey based data as well neuro-response data in order to obtain deeper insights on subject decision making processes. In some implementations, survey based scores and neuro-response data scores are added to obtain an aggregate score. In other examples, survey based scores and neuro-response data scores are scaled and then added to obtain an aggregate score. In still other implementations, scores are scaled and averaged to obtain an aggregate score. However, it is recognized that some of these scores do not accurately reflect DCM evaluations. The techniques and mechanisms of the present invention recognize that survey based measurements and neuro-response based measurements should be treated as separate measurements.

According to various embodiments, survey based measurements and neuro-response based measurements are treated as orthogonal vectors. In particular embodiments, combination of the orthogonal vectors entails scaling and determining the combined magnitude of the vectors using Euclidean geometry and/or linear algebra.

In particular embodiments, subjects are exposed to stimulus material associated with discrete choice modeling and associated choices and data such as central nervous system, autonomic nervous system, and effector data is collected during exposure. According to various embodiments, data is collected in order to determine a resonance measure that aggregates multiple component measures that assess resonance data. In particular embodiments, specific event related potential (ERP) analyses and/or event related power spectral perturbations (ERPSPs) are evaluated for different regions of the brain both before a subject is exposed to stimulus and each time after the subject is exposed to stimulus.

According to various embodiments, pre-stimulus and post-stimulus differential as well as target and distracter differential measurements of ERP time domain components at multiple regions of the brain are determined (DERP). Event related time-frequency analysis of the differential response to assess the attention, emotion and memory retention (DERPSPs) across multiple frequency bands including but not limited to theta, alpha, beta, gamma and high gamma is performed. In particular embodiments, single trial and/or averaged DERP and/or DERPSPs can be used to enhance the resonance measure and determine priming levels for various products and services.

A variety of decision making processes can be analyzed. According to various embodiments, enhanced neuro-response data is generated using a data analyzer that performs both intra-modality measurement enhancements and cross-modality measurement enhancements. According to various embodiments, brain activity is measured not just to determine the regions of activity, but to determine interactions and types of interactions between various regions. The techniques and mechanisms of the present invention recognize that interactions between neural regions support orchestrated and organized behavior. Attention, emotion, memory, and other abilities are not merely based on one part of the brain but instead rely on network interactions between brain regions.

The techniques and mechanisms of the present invention further recognize that different frequency bands used for multi-regional communication can be indicative of the effectiveness of stimuli. In particular embodiments, evaluations are calibrated to each subject and synchronized across subjects. In particular embodiments, templates are created for subjects to create a baseline for measuring pre and post stimulus differentials. According to various embodiments, stimulus generators are intelligent and adaptively modify specific parameters such as exposure length and duration for each subject being analyzed.

A variety of modalities can be used including EEG, GSR, EKG, pupillary dilation, EOG, eye tracking, facial emotion encoding, reaction time, etc. Individual modalities such as EEG are enhanced by intelligently recognizing neural region communication pathways. Cross modality analysis is enhanced using a synthesis and analytical blending of central nervous system, autonomic nervous system, and effector signatures. Synthesis and analysis by mechanisms such as time and phase shifting, correlating, and validating intra-modal determinations allow generation of a composite output characterizing the significance of various data responses.

According to various embodiments, survey based and actual expressed responses and actions for particular groups of users are integrated with neuro-response data and stored in a DCM repository. According to particular embodiments, pre-articulation predictions of expressive response for various stimulus material can be made by analyzing neuro-response data.

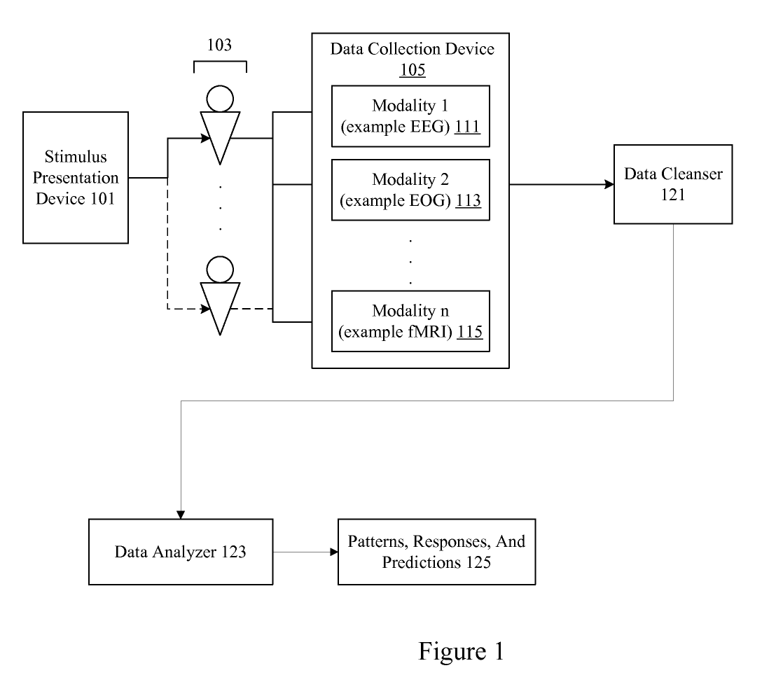

FIG. 1 illustrates one example of a system for performing discrete choice modeling using central nervous system, autonomic nervous system, and/or effector measures.

According to various embodiments, the DCM system includes a stimulus presentation device 101. In particular embodiments, the stimulus presentation device 101 is merely a display, monitor, screen, etc., that displays stimulus material to a user. The stimulus material may be a media clip, a commercial, pages of text, advertisement, etc., that presents multiple options to a subject. The stimuli can involve a variety of senses and occur with or without human supervision. Continuous and discrete modes are supported. According to various embodiments, the stimulus presentation device 101 also has protocol generation capability to allow intelligent customization of stimuli provided to multiple subjects in different markets.

According to various embodiments, stimulus presentation device 101 could include devices such as televisions, cable consoles, computers and monitors, projection systems, display devices, speakers, tactile surfaces, etc., for presenting the stimuli including but not limited to advertising and entertainment from different networks, local networks, cable channels, syndicated sources, websites, internet content aggregators, portals, service providers, etc.

According to various embodiments, the subjects 103 are connected to data collection devices 105. The data collection devices 105 may include a variety of neuro-response measurement mechanisms including neurological and neurophysiological measurements systems such as EEG, EOG, MEG, EKG, pupillary dilation, eye tracking, facial emotion encoding, and reaction time devices, etc. According to various embodiments, neuro-response data includes central nervous system, autonomic nervous system, and effector data. In particular embodiments, the data collection devices 105 include EEG 111, EOG 113, and fMRI 115. In some instances, only a single data collection device is used. Data collection may proceed with or without human supervision.

The data collection device 105 collects neuro-response data from multiple sources. This includes a combination of devices such as central nervous system sources (EEG), autonomic nervous system sources (GSR, EKG, pupillary dilation), and effector sources (EOG, eye tracking, facial emotion encoding, reaction time). In particular embodiments, data collected is digitally sampled and stored for later analysis. In particular embodiments, the data collected could be analyzed in real-time. According to particular embodiments, the digital sampling rates are adaptively chosen based on the neurophysiological and neurological data being measured.

In one particular embodiment, the DCM system includes EEG 111 measurements made using scalp level electrodes, EOG 113 measurements made using shielded electrodes to track eye data, fMRI 115 measurements performed using a differential measurement system, a facial muscular measurement through shielded electrodes placed at specific locations on the face, and a facial affect graphic and video analyzer adaptively derived for each individual.

In particular embodiments, the data collection devices are clock synchronized with a stimulus presentation device 101. In particular embodiments, the data collection devices 105 also include a condition evaluation subsystem that provides auto triggers, alerts and status monitoring and visualization components that continuously monitor the status of the subject, data being collected, and the data collection instruments. The condition evaluation subsystem may also present visual alerts and automatically trigger remedial actions. According to various embodiments, the data collection devices include mechanisms for not only monitoring subject neuro-response to stimulus materials, but also include mechanisms for identifying and monitoring the stimulus materials. For example, data collection devices 105 may be synchronized with a set-top box to monitor channel changes. In other examples, data collection devices 105 may be directionally synchronized to monitor when a subject is no longer paying attention to stimulus material. In still other examples, the data collection devices 105 may receive and store stimulus material generally being viewed by the subject, whether the stimulus is a program, a commercial, printed material, or a scene outside a window. The data collected allows analysis of neuro-response information and correlation of the information to actual stimulus material and not mere subject distractions.

According to various embodiments, the DCM system also includes a data cleanser device 121. In particular embodiments, the data cleanser device 121 filters the collected data to remove noise, artifacts, and other irrelevant data using fixed and adaptive filtering, weighted averaging, advanced component extraction (like PCA, ICA), vector and component separation methods, etc. This device cleanses the data by removing both exogenous noise (where the source is outside the physiology of the subject, e.g. a phone ringing while a subject is viewing a video) and endogenous artifacts (where the source could be neurophysiological, e.g. muscle movements, eye blinks, etc.).

The artifact removal subsystem includes mechanisms to selectively isolate and review the response data and identify epochs with time domain and/or frequency domain attributes that correspond to artifacts such as line frequency, eye blinks, and muscle movements. The artifact removal subsystem then cleanses the artifacts by either omitting these epochs, or by replacing these epoch data with an estimate based on the other clean data (for example, an EEG nearest neighbor weighted averaging approach).

According to various embodiments, the data cleanser device 121 is implemented using hardware, firmware, and/or software. It should be noted that although a data cleanser device 121 is shown located after a data collection device 105, the data cleanser device 121 like other components may have a location and functionality that varies based on system implementation. For example, some systems may not use any automated data cleanser device whatsoever while in other systems, data cleanser devices may be integrated into individual data collection devices.

In particular embodiments, a survey and interview system collects and integrates user survey and interview responses to combine with neuro-response data to more effectively perform DCM. According to various embodiments, the survey and interview system obtains information about user characteristics such as age, gender, income level, location, interests, buying preferences, hobbies, etc. The survey and interview system can also be used to obtain user responses about particular pieces of stimulus material and decision making processes. Scores and weights may be assigned to particular characteristics or features of a product or service based on DCM. In some examples, DCM may be used to determine that energy efficiency improvements rather than aesthetics improvements for a refrigerator would more likely persuade buyers in a particular demographic to make a purchase. In another example, placement of an advertisement for a beverage behind a counter may be less effective than placement of the advertisement in front of a restaurant based on DCM. In still another example, a buyer in a particular demographic group is more likely to select a less expensive lower powered vehicle than a more expensive higher powered vehicle. DCM using both survey based and neuro-response based measurements can be used to quantify the effects of various choices on user behavior.

According to various embodiments, the DCM system includes a data analyzer 123 associated with the data cleanser 121. The data analyzer 123 uses a variety of mechanisms to analyze underlying data in the system to determine resonance. According to various embodiments, the data analyzer 123 customizes and extracts the independent neurological and neuro-physiological parameters for each individual in each modality, and blends the estimates within a modality as well as across modalities to elicit an enhanced response to the presented stimulus material. In particular embodiments, the data analyzer 123 aggregates the response measures across subjects in a dataset.

According to various embodiments, neurological and neuro-physiological signatures are measured using time domain analyses and frequency domain analyses. Such analyses use parameters that are common across individuals as well as parameters that are unique to each individual. The analyses could also include statistical parameter extraction and fuzzy logic based attribute estimation from both the time and frequency components of the synthesized response.

In some examples, statistical parameters used in a blended effectiveness estimate include evaluations of skew, peaks, first and second moments, distribution, as well as fuzzy estimates of attention, emotional engagement and memory retention responses.

According to various embodiments, the data analyzer 123 may include an intra-modality response synthesizer and a cross-modality response synthesizer. In particular embodiments, the intra-modality response synthesizer is configured to customize and extract the independent neurological and neurophysiological parameters for each individual in each modality and blend the estimates within a modality analytically to elicit an enhanced response to the presented stimuli. In particular embodiments, the intra-modality response synthesizer also aggregates data from different subjects in a dataset.

According to various embodiments, the cross-modality response synthesizer or fusion device blends different intra-modality responses, including raw signals and signals output. The combination of signals enhances the measures of effectiveness within a modality. The cross-modality response fusion device can also aggregate data from different subjects in a dataset.

According to various embodiments, the data analyzer 123 also includes a composite enhanced effectiveness estimator (CEEE) that combines the enhanced responses and estimates from each modality to provide a blended estimate of the effectiveness. In particular embodiments, blended estimates are provided for each exposure of a subject to stimulus materials. The blended estimates are evaluated over time to assess resonance characteristics. According to various embodiments, numerical values are assigned to each blended estimate. The numerical values may correspond to the intensity of neuro-response measurements, the significance of peaks, the change between peaks, etc. Higher numerical values may correspond to higher significance in neuro-response intensity. Lower numerical values may correspond to lower significance or even insignificant neuro-response activity. In other examples, multiple values are assigned to each blended estimate. In still other examples, blended estimates of neuro-response significance are graphically represented to show changes after repeated exposure.

According to various embodiments, a data analyzer 123 passes data to a resonance estimator that assesses and extracts resonance patterns. In particular embodiments, the resonance estimator determines entity positions in various stimulus segments and matches position information with eye tracking paths while correlating saccades with neural assessments of attention, memory retention, and emotional engagement. In particular embodiments, the resonance estimator stores data in the priming repository system. As with a variety of the components in the system, various repositories can be co-located with the rest of the system and the user, or could be implemented in remote locations.

Data from various repositories is blended and passed to a DCM engine to generate patterns, responses, and predictions 125. In some embodiments, the DCM engine compares patterns and expressions associated with prior users to predict expressions of current users. According to various embodiments, patterns and expressions are combined with orthogonal survey, demographic, and preference data. In particular embodiments linguistic, perceptual, and/or motor responses are elicited and predicted. Response expression selection and pre-articulation prediction of expressive responses are also evaluated.

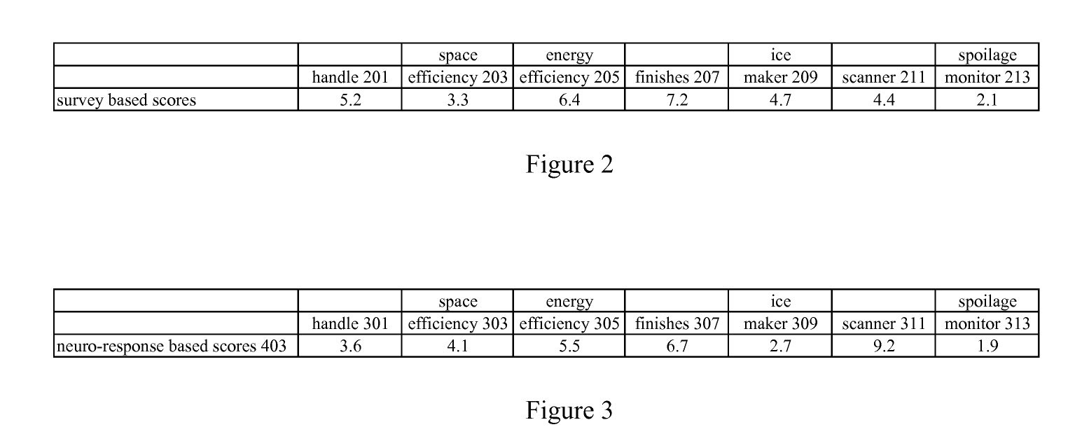

FIG. 2 illustrates survey based scores corresponding to the effectiveness of improving particular features on appliance. The scores may correlate with the propensity of users to desire or purchase the appliance. According to various embodiments, an appliance manufacturer uses discrete choice modeling to determine what feature on an appliance to improve or advertise. Choices of features on a refrigerator may include an improved handle 201, space efficiency 203, energy efficiency 205, finishes 207, ice maker 209, product bar code scanner 211, food spoilage monitor 213, etc. Scores may correspond to the likelihood a user in a particular demographic would purchase the refrigerator if the feature were improved. According to various embodiments, survey based responses are used to determine scores on the scale of 1-10 of 5.2, 3.3, 6.4, 7.2, 4.7, 4.4, and 2.1 corresponding to the features improved handle 201, space efficiency 203, energy efficiency 205, brushed nickel finishes 207, automatic ice cream maker 209, product bar code scanner 211, and food spoilage monitor 213.

FIG. 3 illustrates neuro-response based scores corresponding to the effectiveness of improving particular features on an appliance. The scores may correlate with the propensity of users to desire or purchase the appliance. According to various embodiments, an appliance manufacturer uses discrete choice modeling to determine what feature on an appliance to improve or advertise. Choices of features on a refrigerator may include an improved handle 301, space efficiency 303, energy efficiency 305, finishes 307, ice maker 309, product bar code scanner 311, food spoilage monitor 313, etc. Scores may correspond to the likelihood a user in a particular demographic would purchase the refrigerator if the feature were improved. According to various embodiments, survey based responses are used to determine scores on the scale of 1-10 of 3.6, 4.1, 5.5, 6.7, 2.7, 9.2, and 1.9 corresponding to the features improved handle 301, space efficiency 303, energy efficiency 305, brushed nickel finishes 307, automatic ice cream maker 309, product bar code scanner 311, and food spoilage monitor 313. Although the scores for the neuro-response data and the scores for the survey based data have the same scale in this example, in some instances, scores will have to be converted to the same scale. The scores may be determined using neuro-response data including EEG and eye tracking data.

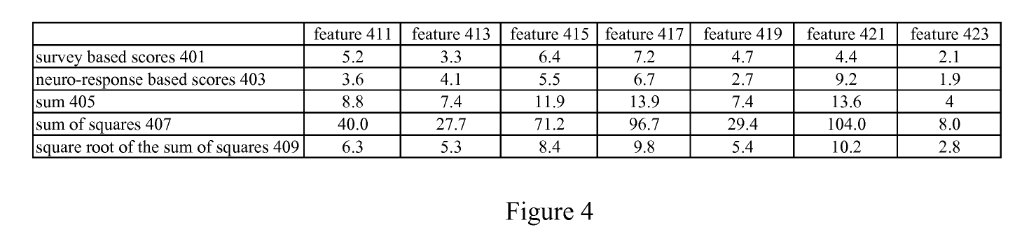

FIG. 4 illustrates types of combinations that can be performed to aggregate survey based data and neuro-response data for DCM. Survey based scores 401 are determined to be 5.2, 3.3. 6.4, 7.2, 4.7, 4.4, and 2.1 for features 411, 413, 415, 417, 419, 421, and 423 respectively. Based on surveys based scores, an evaluator may elect to improve feature 417. Neuro-response based scores 403 are determined to be 3.6, 4.1, 5.5, 6.7, 2.7, 9.2, and 1.9 for features 411, 413, 415, 417, 419, 421, and 423 respectively. Based on neuro-response scores, an evaluator may elect to improve feature 421.

In order to improve insight, evaluators have sought mechanisms of combining survey based scores and statistical scores with neuro-response based scores. In some examples, evaluators simply add or average the scores after scaling. Sums 405 are determined to be 8.8, 7.4, 11.9, 13.9, 7.4, 13.6, and 4 for features 411, 413, 415, 417, 419, 421, and 423 respectively. Based on the summation scores, an evaluator may elect to improve feature 419 based on the highest score 13.9.

According to various embodiments, the techniques and mechanisms of the present invention recognize that survey based scores and neuro-response based scores are separate, orthogonal measurements. To effectively account for the survey based scores and the neuro-response based scores, mechanisms such as Euclidean geometry and/or linear algebra can be used to determine distance between a survey based score vector and a neuro-response response based score vector. Euclidean geometry and linear algebra can be used to determine distance between vectors. Sum of squares 407 are determined to be 40.0, 27.7, 71.2, 96.7, 29.4, 104.0, and 8.0 for features 411, 413, 415, 417, 419, 421, and 423 respectively. Based on the sum of squares 407, feature 421 may be selected. It should be noted the feature selected using sum of squares 407 may be different from the feature selected using mere sums 405.

The square roots of the sum of squares 409 are determined to be 6.3, 5.3, 8.4, 9.8, 5.4, 10.2, and 2.8 for features 411, 413, 415, 417, 419, 421, and 423 respectively. Based on the square root of the sum of squares 409, feature 421 is selected. According to various embodiments, the actual score used is the multi-dimensional distance between the neuro-response data vector and the statistical and/or survey based vector.

In some examples, additional types of data such as statistical data can also be combined using square roots of the sum of squares to determine accurate scores for various features.

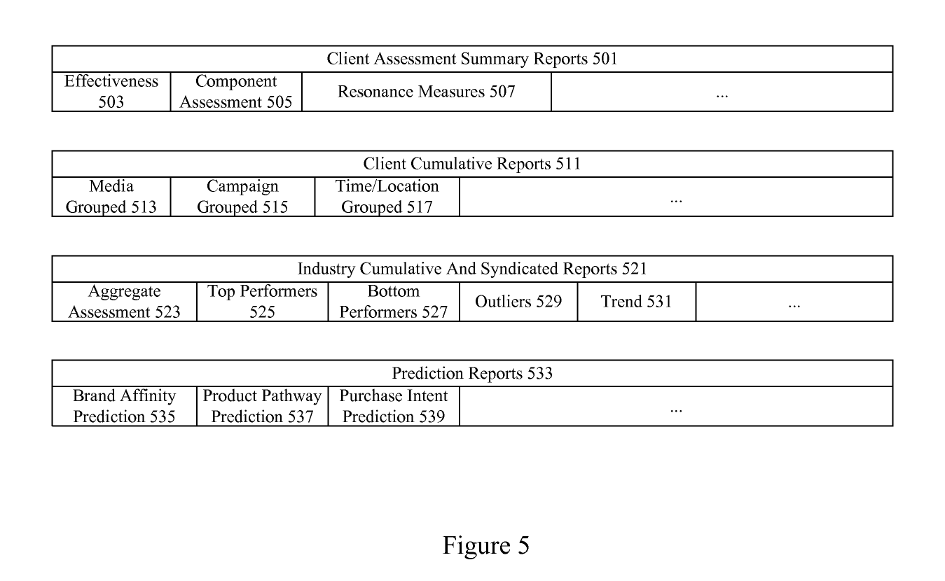

FIG. 5 illustrates examples of reports that can be generated. According to various embodiments, client assessment summary reports 501 include effectiveness measures 503, component assessment measures 505, and resonance measures 507. Effectiveness assessment measures include composite assessment measure(s), industry/category/client specific placement (percentile, ranking, etc.), actionable grouping assessment such as removing material, modifying segments, or fine tuning specific elements, etc, and the evolution of the effectiveness profile over time. In particular embodiments, component assessment reports include component assessment measures like attention, emotional engagement scores, percentile placement, ranking, etc. Component profile measures include time based evolution of the component measures and profile statistical assessments. According to various embodiments, reports include the number of times material is assessed, attributes of the multiple presentations used, evolution of the response assessment measures over the multiple presentations, and usage recommendations.

According to various embodiments, client cumulative reports 511 include media grouped reporting 513 of all stimulus assessed, campaign grouped reporting 515 of stimulus assessed, and time/location grouped reporting 517 of stimulus assessed. According to various embodiments, industry cumulative and syndicated reports 521 include aggregate assessment responses measures 523, top performer lists 525, bottom performer lists 527, outliers 529, and trend reporting 531. In particular embodiments, tracking and reporting includes specific products, categories, companies, brands. According to various embodiments, prediction reports 533 are also generated. Prediction reports may include brand affinity prediction 535, product pathway prediction 537, and purchase intent prediction 539.

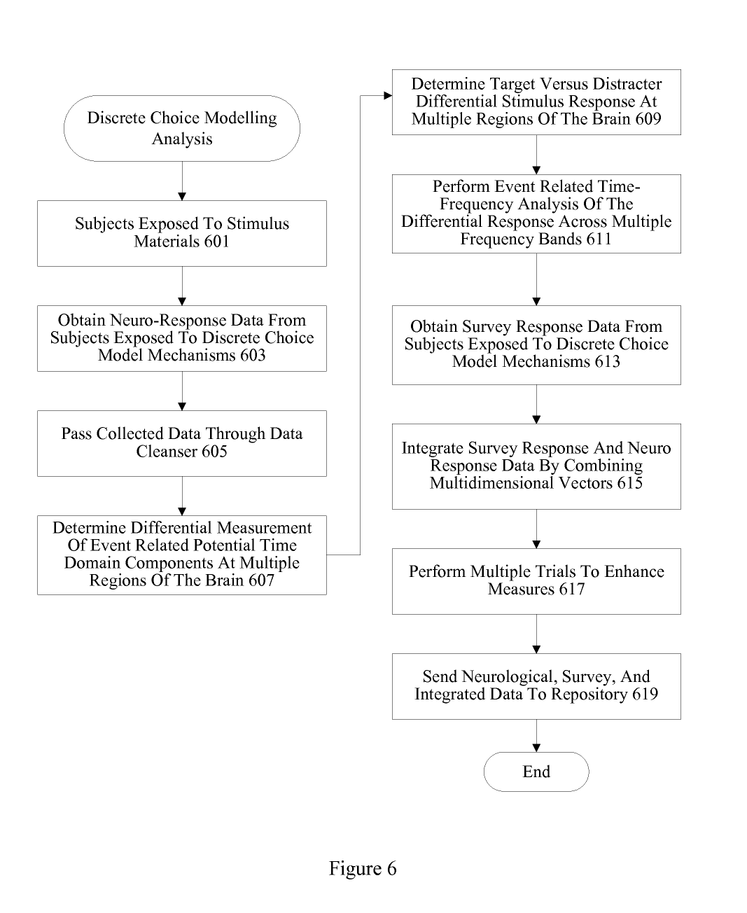

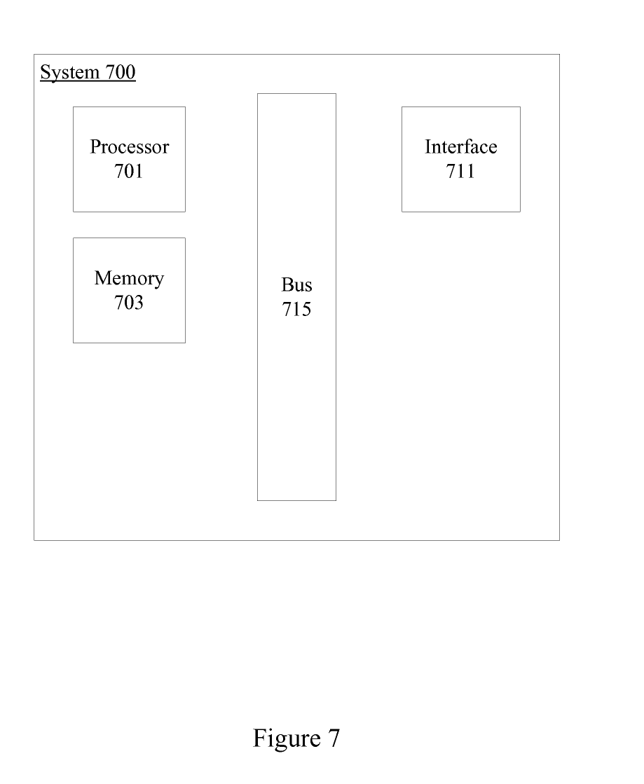

FIG. 6 illustrates one example of DCM. At 601, stimulus material is provided to multiple subjects. According to various embodiments, stimulus includes streaming video and audio. In particular embodiments, subjects view stimulus in their own homes in group or individual settings. In some examples, verbal and written responses are collected for use without neuro-response measurements. In other examples, verbal and written responses are correlated with neuro-response measurements. At 603, subject neuro-response measurements are collected from subjects exposed to discrete choice model mechanisms. In particular embodiments, neuro-response data is collected using a variety of modalities, such as EEG, ERP, EOG, GSR, etc. At 605, data is passed through a data cleanser to remove noise and artifacts that may make data more difficult to interpret. According to various embodiments, the data cleanser removes EEG electrical activity associated with blinking and other endogenous/exogenous artifacts.

According to various embodiments, data analysis is performed. Data analysis may include intra-modality response synthesis and cross-modality response synthesis to enhance effectiveness measures. It should be noted that in some particular instances, one type of synthesis may be performed without performing other types of synthesis. For example, cross-modality response synthesis may be performed with or without intra-modality synthesis.

A variety of mechanisms can be used to perform data analysis. In particular embodiments, a stimulus attributes repository is accessed to obtain attributes and characteristics of the stimulus materials, along with purposes, intents, objectives, etc. In particular embodiments, EEG response data is synthesized to provide an enhanced assessment of effectiveness. According to various embodiments, EEG measures electrical activity resulting from thousands of simultaneous neural processes associated with different portions of the brain. EEG data can be classified in various bands. According to various embodiments, brainwave frequencies include delta, theta, alpha, beta, and gamma frequency ranges. Delta waves are classified as those less than 4 Hz and are prominent during deep sleep. Theta waves have frequencies between 3.5 to 7.5 Hz and are associated with memories, attention, emotions, and sensations. Theta waves are typically prominent during states of internal focus.

Alpha frequencies reside between 7.5 and 13 Hz and typically peak around 10 Hz. Alpha waves are prominent during states of relaxation. Beta waves have a frequency range between 14 and 30 Hz. Beta waves are prominent during states of motor control, long range synchronization between brain areas, analytical problem solving, judgment, and decision making Gamma waves occur between 30 and 60 Hz and are involved in binding of different populations of neurons together into a network for the purpose of carrying out a certain cognitive or motor function, as well as in attention and memory. Because the skull and dermal layers attenuate waves in this frequency range, brain waves above 75-80 Hz are difficult to detect and are often not used for stimuli response assessment.

However, the techniques and mechanisms of the present invention recognize that analyzing high gamma band (kappa-band: Above 60 Hz) measurements, in addition to theta, alpha, beta, and low gamma band measurements, enhances neurological attention, emotional engagement and retention component estimates. In particular embodiments, EEG measurements including difficult to detect high gamma or kappa band measurements are obtained, enhanced, and evaluated. Subject and task specific signature sub-bands in the theta, alpha, beta, gamma and kappa bands are identified to provide enhanced response estimates. According to various embodiments, high gamma waves (kappa-band) above 80 Hz (typically detectable with sub-cranial EEG and/or magnetoencephalograophy) can be used in inverse model-based enhancement of the frequency responses to the stimuli.

Various embodiments of the present invention recognize that particular sub-bands within each frequency range have particular prominence during certain activities. A subset of the frequencies in a particular band is referred to herein as a sub-band. For example, a sub-band may include the 40-45 Hz range within the gamma band. In particular embodiments, multiple sub-bands within the different bands are selected while remaining frequencies are band pass filtered. In particular embodiments, multiple sub-band responses may be enhanced, while the remaining frequency responses may be attenuated.

An information theory based band-weighting model is used for adaptive extraction of selective dataset specific, subject specific, task specific bands to enhance the effectiveness measure. Adaptive extraction may be performed using fuzzy scaling. Stimuli can be presented and enhanced measurements determined multiple times to determine the variation profiles across multiple presentations. Determining various profiles provides an enhanced assessment of the primary responses as well as the longevity (wear-out) of the marketing and entertainment stimuli. The synchronous response of multiple individuals to stimuli presented in concert is measured to determine an enhanced across subject synchrony measure of effectiveness. According to various embodiments, the synchronous response may be determined for multiple subjects residing in separate locations or for multiple subjects residing in the same location.

Although a variety of synthesis mechanisms are described, it should be recognized that any number of mechanisms can be applied—in sequence or in parallel with or without interaction between the mechanisms.

Although intra-modality synthesis mechanisms provide enhanced significance data, additional cross-modality synthesis mechanisms can also be applied. A variety of mechanisms such as EEG, Eye Tracking, GSR, EOG, and facial emotion encoding are connected to a cross-modality synthesis mechanism. Other mechanisms as well as variations and enhancements on existing mechanisms may also be included. According to various embodiments, data from a specific modality can be enhanced using data from one or more other modalities. In particular embodiments, EEG typically makes frequency measurements in different bands like alpha, beta and gamma to provide estimates of significance. However, the techniques of the present invention recognize that significance measures can be enhanced further using information from other modalities.

For example, facial emotion encoding measures can be used to enhance the valence of the EEG emotional engagement measure. EOG and eye tracking saccadic measures of object entities can be used to enhance the EEG estimates of significance including but not limited to attention, emotional engagement, and memory retention. According to various embodiments, a cross-modality synthesis mechanism performs time and phase shifting of data to allow data from different modalities to align. In some examples, it is recognized that an EEG response will often occur hundreds of milliseconds before a facial emotion measurement changes. Correlations can be drawn and time and phase shifts made on an individual as well as a group basis. In other examples, saccadic eye movements may be determined as occurring before and after particular EEG responses. According to various embodiments, time corrected GSR measures are used to scale and enhance the EEG estimates of significance including attention, emotional engagement and memory retention measures.

Evidence of the occurrence or non-occurrence of specific time domain difference event-related potential components (like the DERP) in specific regions correlates with subject responsiveness to specific stimulus. According to various embodiments, ERP measures are enhanced using EEG time-frequency measures (ERPSP) in response to the presentation of the marketing and entertainment stimuli. Specific portions are extracted and isolated to identify ERP, DERP and ERPSP analyses to perform. In particular embodiments, an EEG frequency estimation of attention, emotion and memory retention (ERPSP) is used as a co-factor in enhancing the ERP, DERP and time-domain response analysis.

EOG measures saccades to determine the presence of attention to specific objects of stimulus. Eye tracking measures the subject's gaze path, location and dwell on specific objects of stimulus. According to various embodiments, EOG and eye tracking is enhanced by measuring the presence of lambda waves (a neurophysiological index of saccade effectiveness) in the ongoing EEG in the occipital and extra striate regions, triggered by the slope of saccade-onset to estimate the significance of the EOG and eye tracking measures. In particular embodiments, specific EEG signatures of activity such as slow potential shifts and measures of coherence in time-frequency responses at the Frontal Eye Field (FEF) regions that preceded saccade-onset are measured to enhance the effectiveness of the saccadic activity data.

GSR typically measures the change in general arousal in response to stimulus presented. According to various embodiments, GSR is enhanced by correlating EEG/ERP responses and the GSR measurement to get an enhanced estimate of subject engagement. The GSR latency baselines are used in constructing a time-corrected GSR response to the stimulus. The time-corrected GSR response is co-factored with the EEG measures to enhance GSR significance measures.

According to various embodiments, facial emotion encoding uses templates generated by measuring facial muscle positions and movements of individuals expressing various emotions prior to the testing session. These individual specific facial emotion encoding templates are matched with the individual responses to identify subject emotional response. In particular embodiments, these facial emotion encoding measurements are enhanced by evaluating inter-hemispherical asymmetries in EEG responses in specific frequency bands and measuring frequency band interactions. The techniques of the present invention recognize that not only are particular frequency bands significant in EEG responses, but particular frequency bands used for communication between particular areas of the brain are significant. Consequently, these EEG responses enhance the EMG, graphic and video based facial emotion identification.

According to various embodiments, post-stimulus versus pre-stimulus differential measurements of ERP time domain components in multiple regions of the brain (DERP) are measured at multiple regions of the brain at 607. The differential measures give a mechanism for eliciting responses attributable to the stimulus. For example the messaging response attributable to an advertisement or the brand response attributable to multiple brands is determined using pre-resonance and post-resonance estimates

At 609, target versus distracter stimulus differential responses are determined for different regions of the brain (DERP). At 611, event related time-frequency analysis of the differential response (DERPSPs) are used to assess the attention, emotion and memory retention measures across multiple frequency bands. According to various embodiments, the multiple frequency bands include theta, alpha, beta, gamma and high gamma or kappa.

At 613, survey response information is obtained from multiple subjects exposed to discrete choice model mechanisms. According to various embodiments, survey response data is integrated with neuro-response data for large number of subjects in various geographic and demographic groups at 615 using multidimensional vector combination. In particular embodiments, the square root of the sum of squares of scaled scores are determined to combine neuro-response data and survey data. In some examples, statistical data as well as other data are also integrated. At 617, multiple trials may be performed to enhance measurement. At 619, integrated data is sent to a repository.

According to particular example embodiments, a system 700 suitable for implementing particular embodiments of the present invention includes a processor 701, a memory 703, an interface 711, and a bus 715 (e.g., a PCI bus). When acting under the control of appropriate software or firmware, the processor 701 is responsible for such tasks such as pattern generation. Various specially configured devices can also be used in place of a processor 701 or in addition to processor 701. The complete implementation can also be done in custom hardware. The interface 711 is typically configured to send and receive data packets or data segments over a network. Particular examples of interfaces the device supports include host bus adapter (HBA) interfaces, Ethernet interfaces, frame relay interfaces, cable interfaces, DSL interfaces, token ring interfaces, and the like.

According to particular example embodiments, the system 700 uses memory 703 to store data, algorithms and program instructions. The program instructions may control the operation of an operating system and/or one or more applications, for example. The memory or memories may also be configured to store received data and process received data.

Because such information and program instructions may be employed to implement the systems/methods described herein, the present invention relates to tangible, machine readable media that include program instructions, state information, etc. for performing various operations described herein. Examples of machine-readable media include, but are not limited to, magnetic media such as hard disks, floppy disks, and magnetic tape; optical media such as CD-ROM disks and DVDs; magneto-optical media such as optical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

Although the foregoing invention has been described in some detail for purposes of clarity of understanding, it will be apparent that certain changes and modifications may be practiced within the scope of the appended claims. Therefore, the present embodiments are to be considered as illustrative and not restrictive and the invention is not to be limited to the details given herein, but may be modified within the scope and equivalents of the appended claims.

What is claimed is:

1. A method, comprising:
exposing a plurality of subjects to stimulus material associated with a discrete choice model;
obtaining neuro-response data from the plurality of subjects exposed to the stimulus material;
generating a plurality of neuro-response scores corresponding to a plurality of choices in the discrete choice model;
obtaining survey response data;
generating a plurality of survey response scores corresponding to the plurality of choices in the discrete choice model;
aggregating the neuro-response scores and the survey response scores using mult-dimensional vector combination.

2. The method of claim 1, wherein multi-dimensional vector combination comprises computing the square of the sum of squares for corresponding neuro-response scores and survey response scores.

3. The method of claim 1, wherein multi-dimensional vector combination comprises computing the combined magnatitude of neuro-response score vectors and corresponding survey response score vectors.

4. The method of claim 1, wherein a plurality of neuro-response score vectors are orthogonal to a plurality of survey response score vectors.

5. The method of claim 1, wherein choices correpsond to features of a products or service.

6. The method of claim 1, wherein neuro-response data is collected using a plurality of modalities including Electronencephalography (EEG) and Electrooculography (EOG).

7. The method of claim 1, wherein obtaining neuro-response data comprises obtaining target and distracter event related potential (ERP) measurements to determine differential measurements of ERP time domain components at multiple regions of the brain (DERP).

8. The method of claim 1, wherein obtaining neuro-response data further comprises obtaining event related time-frequency analysis of the differential response to assess the attention, emotion and memory retention (DERPSPs) across multiple frequency bands.

9. The method of claim 1, wherein survey response data is obtained from the plurality of subjects exposed to stimulus material.

10. The method of claim 1, wherein the survey response scores and the neuro-response scores are scaled prior to combination.

11. A system, comprising:
a data collection mechanisms operable to obtain neuro-response data from a plurality of subjects exposed to the stimulus material associated with a discrete choice model and operable to otain survey response data for the stimulus material;
a data analyzer operable to generate a plurality of neuro-response scores corresponding to a plurality of choices in the discrete choice model and a plurality of survey response scores corresponding to the plurality of choices in the discrete choice model;
wherein the neuro-response scores and the survey response scores are aggregated using mult-dimensional vector combination.

12. The system of claim 11, wherein multi-dimensional vector combination comprises computing the square of the sum of squares for corresponding neuro-response scores and survey response scores.

13. The system of claim 11, wherein multi-dimensional vector combination comprises computing the combined magnatitude of neuro-response score vectors and corresponding survey response score vectors.

14. The system of claim 11, wherein a plurality of neuro-response score vectors are orthogonal to a plurality of survey response score vectors.

15. The system of claim 11, wherein choices correpsond to features of a products or service.

16. The system of claim 11, wherein neuro-response data is collected using a plurality of modalities including Electronencephalography (EEG) and Electrooculography (EOG).

17. The system of claim 11, wherein obtaining neuro-response data comprises obtaining target and distracter event related potential (ERP) measurements to determine differential measurements of ERP time domain components at multiple regions of the brain (DERP).

18. The system of claim 11, wherein obtaining neuro-response data further comprises obtaining event related time-frequency analysis of the differential response to assess the attention, emotion and memory retention (DERPSPs) across multiple frequency bands.

19. The system of claim 11, wherein survey response data is obtained from the plurality of subjects exposed to stimulus material.

20. The system of claim 11, wherein the survey response scores and the neuro-response scores are scaled prior to combination.

21. An apparatus, comprising:
means for exposing a plurality of subjects to stimulus material associated with a discrete choice model;
means for obtaining neuro-response data from the plurality of subjects exposed to the stimulus material;
means for generating a plurality of neuro-response scores corresponding to a plurality of choices in the discrete choice model;
means for obtaining survey response data;
means for generating a plurality of survey response scores corresponding to the plurality of choices in the discrete choice model;
means for aggregating the neuro-response scores and the survey response scores using mult-dimensional vector combination.
